(19) United States
(12) Patent Application Publication
(10) Pub. No.: US 2025/0153315 A1
(43) Pub. Date: May 15, 2025

(54) WRENCH WITH TORQUE INDICATOR

(71) Applicant: Bosonic AG, Bern (CH)

(72) Inventors: Steve Knuchel, Biel/Bienne (CH); Clémentine Favre, Echichens (CH); Martin Sigrist, Bern (CH); Samuel Malzach, Evillard (CH); Thomas Kaupert, Muri b. Bern (CH)

(21) Appl. No.: 18/841,096

(22) PCT Filed: Feb. 28, 2023
(86) PCT No.: PCT/IB2023/051837

§ 371 Date: Aug. 23, 2024

(30) Foreign Application Priority Data
Mar. 2, 2022 (CH) 000207/2022

(51) Int. Cl. B25B 13/08; B25B 23/142

(52) U.S. Cl. CPC B25B 13/08; B25B 23/1427

(57) ABSTRACT

A monolithic wrench for indicating if an applied torque is within a predefined range, including a handle formed in one single piece, a head for accommodating an element to tighten or loosen with the wrench, an outer contour defining the maximal dimensions of the wrench, and a torque range indicator accommodated in an inner opening entirely provided within the outer contour. The torque range indicator includes an amplification mechanism for amplifying deformations of the head relative to the handle when the wrench is actuated, and includes an indicating element on the amplification mechanism for indicating a range of torque based on a relative displacement of the head with respect to the handle.

### BACKGROUND OF THE INVENTION

### Field of the Invention

The present invention concerns a wrench configured to indicate a range of an applied torque.

### Description of Related Art

Wrench tools with torque indicator are widely known in the art. The torque indicators can be mechanical or digital and achieve high precision of the torque measurement.

In specific fields and situations, it may be more important for a user to know that an applied torque does not exceed a predefined threshold or is in a particular range, than to know the exact amplitude. For example, some cutting devices used for surgical purposes have a cutting tool that can be screwed into a handpiece. If the tool is not tightened enough, it might loosen and disrupt proper usage of the device. If it is tightened too much, the cutting tool or the device might be damaged due to stress in the material. This could result in the cutting tool breaking or the threads ripping out of the device, which could potentially be dangerous for the patient undergoing the surgery. To prevent these cases, the cutting tool must be tightened in a certain torque range.

Existing designs of torque wrenches that can be used in such situations are often complex and expensive. Indeed, they are usually made out of several components that have to be assembled which leads to many disadvantages such as: complex manufacturing, complex maintenance (e.g. sterilization or cleaning), need for lubrification, high risk of contact corrosion, etc.

The document JP2016043448A discloses a wrench whose notched handle is able to indicate a range of an applied torque. This wrench is made out of several assembled components.

An example of monolithic torque wrench is disclosed in the document U.S. Pat. No. 7,597,032. The disclosed torque wrench is actually a ratchet wrench with a two-parts handle acting as a torque range indicator. If a torque is applied in a first rotation sense, a first part of the handle moves away from the second part of the handle. An indicating element fixed to one of the two parts indicates the range of the applied torque. If a torque is applied in the opposite rotation sense, the first part of the handle is pushed against the second part, which acts as a lock mechanism. This two-parts design of the handle can be bulky when the first part of the handle moves away from the second part. Moreover, this design may not allow a firm grip on the handle as the operator has to let the first part free to move away from the second part.

The document JP2016043448A discloses a wrench whose notched handle is able to indicate a range of an applied torque. This wrench is made out of several assembled components.

### SUMMARY OF THE INVENTION

An aim of the present invention is to provide a wrench with a torque indicator that overcomes the shortcomings and limitations of the state of the art.

Another aim of the invention is to provide a wrench with a torque indicator which is more ergonomic, in particular which allows a user to easily read a torque indication while actuating the wrench.

These aims are achieved by the object of the claims and in particular by a monolithic wrench for indicating if an applied torque is within one predefined range, including:
a handle;
a head for accommodating an element to tighten or loosen with the wrench;
an outer contour defining the maximal dimensions of the wrench;
a torque range indicator accommodated in an inner opening entirely comprised within the outer contour, wherein the torque range indicator includes an amplification mechanism for amplifying deformations of the head relative to the handle when the wrench is actuated, and includes an indicating element on the amplification mechanism for indicating a range of torque based on a relative displacement of the head with respect to the handle.

The monolithic design allows the wrench to be simple and easy to manufacture, in particular because it does not require any assembly. It also makes the wrench easier to maintain (e.g. clean, sterilize, no lubrication etc).

The handle can be formed in one single rigid piece permitting an easier grasp of the wrench during its actuation, preventing the movement of the torque range indicator to perturbate the actuation.

A further advantage of this single rigidly formed handle is that a user can easily read the torque indications as the torque indicator is not localized under his hand. The torque range indicator is not disturbed by the user's hand.

The relative displacement of the head with respect to the handle due to the elastic deformation of the used material can be further increased by connecting the head to the handle through at least one flexible beam.

The amplification mechanism may include at least two flexible arms connected through a connecting element and arranged to move away from the connecting element when a first torque is applied to a contact zone in a rotation sense and to move toward the connecting element when a second torque is applied to the contact zone in the opposite rotation sense.

The at least one flexible beam and the connecting element allow the amplification mechanism to further deform and increase the range of torque indication.

The wrench may include a torque range marking. The position of the indicating element relatively to this torque range marking being representative of a corresponding torque.

The torque range marking may be disposed on the handle and the indicating element may point toward said handle.

The wrench may be made of a metal alloy, preferably a steel-based alloy, an aluminium-based alloy, a titanium-based alloy, or of ceramic, polymer, silicon or wood.

The range of torque may be comprised between 0.2 Nm and 200 Nm.

The wrench may be manufactured by subtractive or by additive manufacturing.

The head of the wrench can be configured so that at least two sizes of element to tighten or loosen can be accommodated within the head.

According to an embodiment, the head can include two branches delimiting a void space in-between so as to accommodate an element to tighten or loosen. This void space includes a first portion of a first width for accommodating an element to tighten/loosen with a corresponding width, and a second portion of a second width for accommodating an element to tighten/loosen with a corresponding width.

The first width is typically greater than the second width.

The wrench can be used for tightening and/or loosening an element of a medical tool, the range of the torque being comprised between 0.2 Nm and 10 Nm.

The medical tool can be a surgical cutting tool.

### BRIEF DESCRIPTION OF THE DRAWINGS

Exemplar embodiments of the invention are disclosed in the description and illustrated by the drawings in which:

FIG. 1 illustrates a perspective view of a wrench with a torque indicator.

FIG. 2 illustrates a lateral view of a wrench with a torque indicator.

FIG. 3 illustrates a lateral view of a torque range indicator when no torque is applied.

FIG. 4 illustrates a lateral view of a torque range indicator when a torque is applied to tighten an element.

FIG. 5 illustrates a lateral view of a torque range indicator when a torque is applied to loosen an element.

FIG. 6 illustrates a perspective view of a wrench with a torque indicator including an indicating element pointing towards the head of the wrench.

FIG. 7 illustrates a lateral view of a wrench with a torque indicator including an indicating element pointing towards the head of the wrench.

FIG. 8 illustrates a lateral view of a wrench whose head is adapted to two different sizes of element to tighten or loosen.

### DETAILED DESCRIPTION OF THE INVENTION

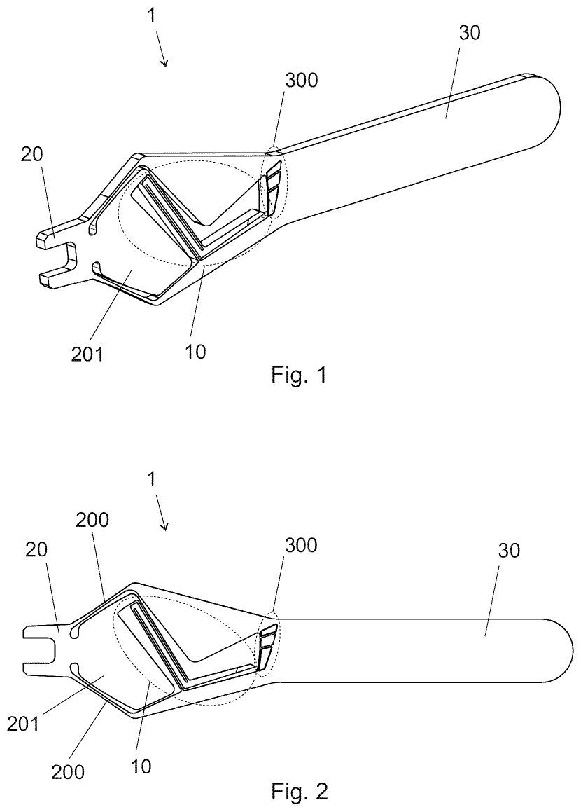

A monolithic wrench 1 according to the invention is illustrated in FIG. 1. The wrench 1 includes a head 20, a handle 30 and a torque range indicator 10. The head 20 can accommodate an element to be tightened or loosened by applying a torque. The handle 30 allows an operator to apply a force on the wrench so as to create a torque. The torque range indicator 10 is a mechanical indicator that is configured to indicate a range of an applied torque so as to allow to an operator to know if the applied torque is within a predefined range.

In the context of this disclosure, the expression “monolithic wrench” means that the wrench is made out of a single piece of material, and in particular does not require any assembly during its manufacturing. This monolithic design can be obtained by traditional machining (i.e. subtractive manufacturing) or alternatively by additive manufacturing (i.e. 3D-printing).

This monolithic design allows an easy and simple maintenance of the wrench as no disassembly is required to clean or lubricate tool parts. Moreover, the risk of corrosion is reduced as a single material is used, avoiding contact corrosion. As illustrated in FIG. 2, the torque range indicator 10 is disposed in an inner opening of the wrench 1, the inner opening being completely comprised in an outer contour of the wrench determining its maximal dimensions. The inner opening may be a through opening or alternatively a blind opening.

Advantageously, the handle 30 is made of one single rigid piece. This means that the handle 30 is integrally formed and does not include arms that can move respectively to one another when the wrench is actuated. This allows a better grasping of the wrench by its user as the whole handle can be held.

In the context of the present disclosure, the term “handle” refers to the portion of the wrench opposed to the head 20 and does not include the torque range indicator 10. The purpose of the handle 30 is to allow the actuation of the wrench.

A handle 30 in one single rigid piece is made possible because the torque range indicator, and more specifically the amplification mechanism, are completely comprised within the outer contour of the wrench. In particular, the torque range indicator does not rely on the relative movement of subparts of the handle 30.

When a torque is applied to the handle 30 by an operator to tighten or loosen an element accommodated in the head 20, the contact zone 201, which connects the head to the handle, undergoes an elastic deformation which can vary according to the material used for manufacturing the wrench. This deformation results in a movement of the head 20 relatively to the handle 30, and is proportional to the applied torque. Therefore, a quantification of this deformation leads to a quantification of the applied torque.

The torque range indicator 10 includes an amplification mechanism 10A for amplifying the deformation of the handle 30 relatively to the head 20 and includes an indicating element 10B for providing an information about the range of the applied torque. The indicating element is able to move in response to the deformation, which allows an operator actuating the wrench to have information about the amplitude of the applied torque.

In one embodiment, the amplification mechanism 10A includes at least two flexible arms that are connected through a rigid connecting element 101 which stabilizes the flexible arms. The flexible arms are arranged to move away from the connecting element when a first torque is applied to the contact zone 201 in a rotation sense and to move toward the connecting element when a second torque is applied to the contact zone in the opposite rotation sense.

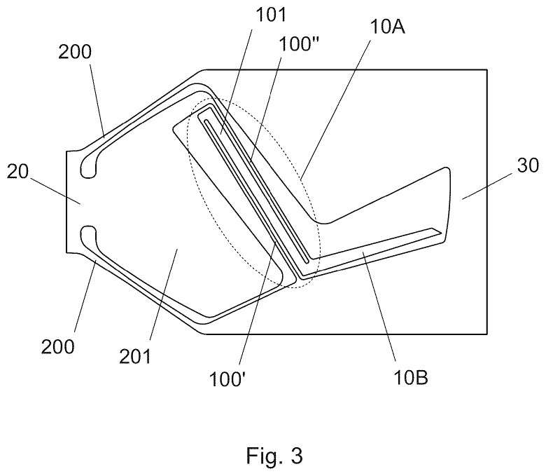

As illustrated in FIG. 3, the amplification mechanism 10A may include a first flexible arm and a second flexible arm 100″. The first flexible arm 100′ is disposed between the contact zone 201 and the connecting element 101, while the second flexible arm 100″ is disposed between the connecting element and the handle 30. The first flexible arm 100′ is attached at one of its extremities to the handle 30 and at the other extremity to a first extremity of the connecting element 101. The second flexible arm 100″ is attached at one of its extremities to the contact zone 201 and at the other extremity to a second extremity of the connecting element 201. When no torque is applied to the wrench, both flexible arms are essentially parallel to the connecting element 101.

In another embodiment not illustrated, the flexible arms and the connecting element are not parallel in the rest position, i.e. in the position in which no torque is applied.

The indicating element 10B is usually fixed to the amplification mechanism 10A so that a movement of the amplification mechanism is transferred to the indicating element. The amplitude of the indicating element movement is then representative of the applied torque.

A torque range marking 300 may be disposed on the wrench so that the relative position of the indicating element 10B relatively to this marking corresponds to an applied torque. Such a torque range marking may be for example a graduated scale, a nonius marking, one or more zones corresponding to particular ranges, etc.

Advantageously, the torque range marking is placed on a portion of the wrench which is not covered by a user's hand actuating the wrench so that the user may easily get the information of the range of applied torque while using the wrench.

In an embodiment, the indicating element 10B includes a needle which has an extremity attached to the connecting element 101. The other extremity of the needle is used as a pointer towards a particular zone of the wrench 1. A torque range marking 300 representative of a torque range applied to the wrench can be added to this particular zone so that the needle points towards the marking and a user can get an information about the applied torque by looking at the marking.

As illustrated in FIG. 2, a torque range marking 300 can be disposed on the handle 30. This marking can consist in several zones, for example three zones, each zone corresponding to a range of torque.

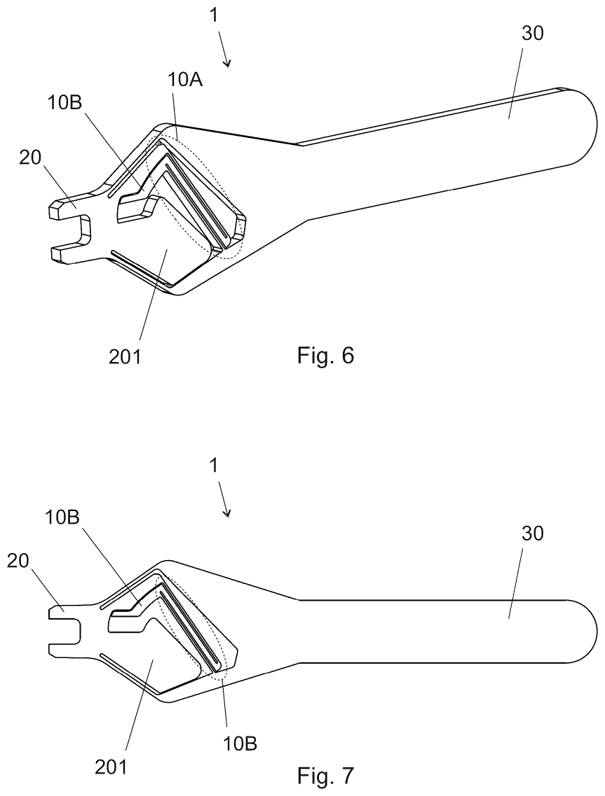

In a preferred embodiment illustrated in FIG. 3, the needle of the indicating element 10B may for example point towards the handle 30. In another embodiment illustrated in FIG. 6, the needle may point towards the head 20.

Given a torque, the relative displacement of the needle respectively to the handle is greater when the needle points towards the handle since the handle moves with respect to the head and in an inverse sense relatively to the needle. However, for design reasons, it may be convenient to have a needle pointing towards the head.

Alternatively or complementarily, the indicating element 10B may also indicate a torque range by masking a designated portion of the wrench, or of the torque range marking.

In another embodiment, the torque range marking includes one or more notches or slots in which the indicating element 10B can fit into according to a corresponding torque, each notch or slot representing a value of an applied torque.

In order to increase the deformation of the head 20 relatively to the handle 30, the wrench 1 may include at least one flexible beam connecting the head to the handle. These flexibles beams allow to increase the elastic deformation. The flexible beams may extend radially from the head 20 so as to align with the theoretical pivot point of the element to be tightened.

In a particular embodiment illustrated in FIG. 2, two flexible beams 200 are disposed symmetrically around the longitudinal axis of the wrench. The contact zone 201 is fixed to the head through a neck, and is able to move within the inner opening which is partially delimited by the flexible beams. This movement of the contact zone results in a significant amplification of the deformation of the wrench which is to be quantified and therefore allows refined measurements of this torque.

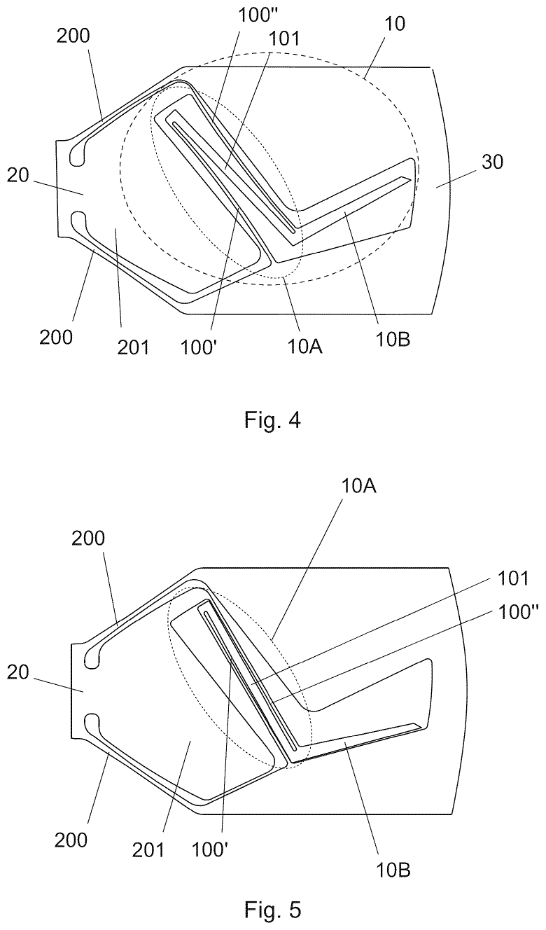

FIG. 4 illustrates the torque range indicator 10 in a configuration obtained when a first torque is applied in a first rotation sense to the contact zone 201, for example in order to tighten an element accommodated in the head 20. The two flexible beams 200 are deformed and the contact zone 201 moves with respect to the handle 30. The movement of the contact zone causes the first flexible arm 100′ to progressively move away from the connecting element 101. The extremity of the first arm that is attached to the connecting element does not move away from it while the extremity of the first arm that is attached to the handle is moved away from the connecting element. Similarly, the second flexible arm 100″ progressively moves away from the connecting element. The movement of the two flexible arms causes the connecting element to rotate in a sense which is opposite to the first rotation sense. The indicating element 10B which is attached to the connecting element 101 is therefore moved in a direction that is essentially opposite to the first rotation sense.

FIG. 5 illustrates the opposite configuration in which a torque is applied in a second rotation sense that opposite to the first rotation sense, for example for loosening an element accommodated in the head 20. In this situation, both the first and the second flexible arms 100′, 100″ are moved towards the connecting element 101 causing its rotation in a sense which is opposite to the second rotation sense. Consequently, the indicating element 10B is moved in a direction that is essentially opposite to the second rotation sense. The indicating element 10B may come into contact with an inner surface of the wrench (i.e. the boundary of the inner opening) and act as a lock-stop, stopping the amplification mechanism to further deform. Alternatively or complementarily, a flexible arm may also act as a lock-stop mechanism when it comes into contact with the connecting element.

In order to be reusable, the wrench has to be manufactured with a material that can handle high stresses without undergoing plastic deformations. Moreover, other external factors such as heat resistance (sterilization) or bio-compatibility can be important for the choice of the material. Indeed, the wrench of the present invention is suitable for use in the medical field, and particularly in the surgical field, for fixing certain surgical tools (such as cutting tools) to a handpiece (or to remove these tools from the handpiece). Such uses require a sterilization of the wrench.

Alternatively, materials with particular plastic deformation properties can be used, for example to obtain single use wrenches that stay deformed after use.

Several different materials may be used for manufacturing the monolithic wrench. Such materials include several types of metal alloys such as steel-based alloys, for example chromium-nickel-copper steel, as well as aluminium-based alloys or titanium-based alloys. Alternatively, materials such as ceramic, different types of polymers, silicon or wood can be considered depending on the intended use of the wrench. The choice of the material may strongly influence the mechanical properties of the wrench. Therefore, it can be used to either increase or decrease the total deformation of the wrench, i.e. the plastic and elastic deformation.

As aforementioned, the wrench 1 may be manufactured by traditional machining. Various cutting techniques can be used to determine the wrench contour and inner opening such as water jet, EDM (electrical discharge machining), laser cutting, etc. Steps of hardening, corrosion treatments, engraving can be applied. In particular, a series number as well as the torque range marking can be engraved by laser on the handle.

The dimensions of the wrench, namely the plate thickness, also strongly influence the range of torque that can be applied. The thickness of the present wrench may advantageously vary between 1 mm and 10 mm according to the range of torque that needs to be achieved. This thickness can be changed according to the desired torque without needing to change other parameters of the design of the wrench.

The head 20 can be configured to accommodate at least two different sizes of elements to tighten or loosen. This means that the same wrench can be used to tighten or loosen elements having at least two different sizes, i.e. at least two different widths.

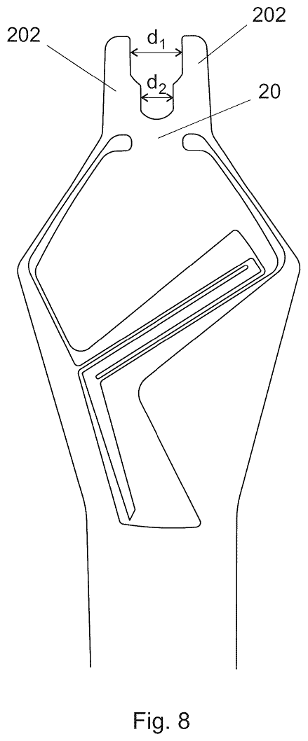

In an embodiment illustrated in FIG. 8, the head 20 includes two branches 202 delimiting a void space between the two branches. This void space is meant to accommodate the elements to be tightened or loosened.

The inner distance between the two branches 202 is variable along the length of the branches so as to permit to accommodate different sizes of elements to tighten or loosen. Consequently, the void space delimited by the branches 202 is of variable width, as its width corresponds to the inner distance between the two branches.

In a particular embodiment, the void space includes a first portion having a first width d₁, that is the two branches include first portions spaced one from another from a distance corresponding to the first width d₁. This first portion of the void space can accommodate elements to be tightened or loosened having a size corresponding to the first width d₁. The void space further includes a second portion having a second width d₂, meaning that the two branches include second portion spaced apart from the first by a distance corresponding to the second width d₂. This second portion of the void space can accommodate elements to be tightened or loosened having a size corresponding to the second width d₂.

In an embodiment not represented, the void space includes more than two portions with different widths so as to allow elements with more than two sizes (i.e. widths) to be tightened or loosened.

As illustrated in FIG. 8, the first width d₁ can be greater than the second width d₂.

### LIST OF REFERENCE NUMERALS

1 Wrench
10 Torque range indicator
10A Amplification mechanism
100′ First flexible arm
100″ Second flexible arm
101 Connecting element
10B Indicating element
20 Head
200 Flexible beam
201 Contact zone
202 Branch
30 Handle
300 Torque range marking
d₁ First width
d₂ Second width

1. A monolithic wrench for indicating if an applied torque is within one predefined range, comprising:
a handle;
a head for accommodating an element to tighten or loosen with said wrench;
an outer contour defining the maximal dimensions of the wrench;
a torque range indicator accommodated in an inner opening entirely comprised within said outer contour, wherein said torque range indicator comprises an amplification mechanism for amplifying deformations of the head relative to the handle when the wrench is actuated, and comprises an indicating element on said amplification mechanism for indicating a range of torque based on a relative displacement of the head with respect to the handle.

2. The monolithic wrench of claim 1, said handle being made of one single rigid piece.

3. The monolithic wrench of claim 1, said head being connected to said handle through at least one flexible beam.

4. The monolithic wrench of claim 1, said amplification mechanism comprising at least two flexible arms connected through a connecting element and arranged to move away from the connecting element when a first torque is applied to a contact zone in a rotation sense and to move toward the connecting element when a second torque is applied to the contact zone in the opposite rotation sense.

5. The monolithic wrench of claim 1, further comprising a torque range marking, a position of the indicating element relatively to the torque range marking being representative of a corresponding torque.

6. The monolithic wrench of claim 5, the torque range marking being disposed on the handle and the indicating element pointing toward said handle.

7. The monolithic wrench of claim 1, wherein said monolithic wrench is made of a steel-based alloy, an aluminium-based alloy or a titanium-based alloy, or of ceramic, polymer, silicon or wood.

8. The monolithic wrench of claim 1, wherein said range of torque is between 0.2 Nm and 200 Nm.

9. The monolithic wrench of claim 1, wherein said monolithic wrench is manufactured by subtractive or by additive manufacturing.

10. The monolithic wrench of claim 1, wherein said head is configured to accommodate at least two different sizes of elements to tighten or loosen.

11. The monolithic wrench of claim 1, said head comprising two branches delimiting a void space between the two branches, said void space comprising a first portion of a first width for accommodating a first element to tighten or loosen and a second portion of a second width for accommodating a second element to tighten or loosen.

12. The monolithic wrench of claim 1, said first width being greater than said second width.

13. A method for tightening and/or loosening an element of a medical tool, comprising the steps of:
providing the monolithic wrench of claim 1;
applying torque to said element of the medical tool with said monolithic wrench, said torque being between 0.2 Nm and 10 Nm.

14. Wrench of claim 13, wherein said medical tool is a surgical cutting tool.
